(19) United States
(12) Patent Application Publication
(10) Pub. No.: US 2026/0218104 A1
(43) Pub. Date: Jul. 30, 2026

(54) Method and Apparatus for Investigating Mammalian Cells

(71) Applicant: Robert Bosch GmbH, Stuttgart (DE)

(72) Inventors: Sonja Kuellmer, Calw (DE); Michael Stumber, Korntal-Muenchingen (DE); Bernd Scheufele, Koengen (DE)

(21) Appl. No.: 19/147,698

(22) PCT Filed: Nov. 16, 2023
(86) PCT No.: PCT/EP2023/081988

§ 371 Date: Jul. 12, 2025

(30) Foreign Application Priority Data
Jan. 16, 2023 (DE) 10 2023 200 275.2

(51) Int. Cl. C12M 3/06; C12M 1/00; C12M 1/34; C12N 5/071; C12N 9/22; C12N 15/11; C12Q 1/6816

(52) U.S. Cl. CPC C12M 23/16; C12M 29/20; C12M 41/46; C12M 47/06; C12N 5/0602; C12N 9/222; C12N 15/11; C12Q 1/6816; C12N 2310/20; C12N 2320/10

(57) ABSTRACT

A microfluidic apparatus is disclosed having a diffusion chamber into which multiple feeds open. At least one first feed opens into the diffusion chamber below at least one second feed. And at least one third feed opens into the diffusion chamber above the second feed. A method for investigating mammalian cells in the microfluidic apparatus includes introducing mammalian cells into the diffusion chamber through the first inlet and lysing the mammalian cells.

The present invention relates to a microfluidic apparatus. Furthermore, the present invention relates to a method for investigating mammalian cells in the microfluidic apparatus.

### PRIOR ART

Models of cancer, such as circulating tumor cells, organoid cultures, and patient-derived transplants, can be used for personalized cancer therapy and drug development.

U.S. Pat. No. 11,098,369 B2 describes a method for determining the viability of tumor cell spheroids in a three-dimensional microfluidic apparatus. Viability can be determined by measuring the total fluorescence.

K. Yin et al., Dynamic aqueous multiphase reaction system for one-pot CRISPR-Cas12a based ultrasensitive and quantitative molecular diagnosis, Analytical Chemistry 2020, pages 8561-8568, describes how quantitative genetic analysis of genomic DNA can be performed using time-resolved fluorescence detection by means of separate reaction sequences for cell lysis, DNA amplification, and CRISPR-Cas12a-based DNA detection in a sucrose gradient.

### DISCLOSURE OF THE INVENTION

The microfluidic apparatus has a diffusion chamber into which several feeds open. At least one first feed opens into the diffusion chamber below at least one second feed. At least one third feed opens into the diffusion chamber above the second feed.

The three feeds are arranged in particular such that, when the device is used as intended, the second feed opens into the diffusion chamber higher than the first feed and the third feed opens into the diffusion chamber higher than the second feed, and thus the three feeds are preferably arranged vertically one above the other.
This microfluidic apparatus can be used in particular to subject cells, preferably mammalian cells, which may originate, for example, from a 3D culture of mammalian tumor cells, to different analysis steps.

The diffusion chamber is preferably made of at least one material selected from the group consisting of polycarbonates (PC), polymethyl methacrylates (PMMA), cycloolefin copolymers (COC), cycloolefin polymers (COP), polystyrenes (PS), and glass. These materials have the advantage of being transparent, thus enabling optical examination of the contents of the diffusion chamber, in particular by means of fluorescence spectroscopy.

The first feed is preferably fluidically connected to a cell culture element and, in particular, to a processing element for separating and splitting mammalian cells from 3D cell cultures. This makes it possible to introduce mammalian cells from a cell culture, in particular from a 3D cell culture, as a cell suspension into the lower region of the diffusion chamber. The second feed and the third feed are each preferably fluidically connected to a reagent reservoir. This makes it possible to overlay the mammalian cells in the diffusion chamber by means of reagents introduced through the second feed and/or the third feed.

In the context of the present invention, the term “splitting” refers to the breaking of connections between cells of a cell agglomerate, such as an organoid or spheroid, and the resulting dissociation of the cell agglomerate into multicellular cell agglomerate fragments or individual cells.

The processing element can, for example, be a chamber or a structure in which separation or splitting takes place.
The diffusion chamber preferably has at least one vent opening on its top surface. In this way, air can be displaced from the diffusion chamber through the vent opening when a cell suspension or reagents are introduced. It is particularly preferred to arrange a filter in the vent opening to prevent contaminants from entering the diffusion chamber through the vent opening.

The first feed opens in particular into a first segment of the diffusion chamber, the second feed opens in particular into a second segment of the diffusion chamber, and the third feed opens in particular into a third segment of the diffusion chamber. Each segment is designed to be filled with a different fluid. In principle, the segments cannot be sections of the diffusion chamber that are separated from one another. However, it is preferable that a web is arranged between the first segment and the second segment and between the second segment and the third segment. The webs run horizontally in particular and are arranged immediately below the second feed and immediately below the third feed in particular. They enable improved capillary filling of the diffusion chamber and separate the segments from one another.

In one embodiment of the microfluidic apparatus, the third segment is divided into several subsegments by means of at least one partition wall. This partition wall runs in particular vertically. A third feed opens into each subsegment. This makes it possible to fill each subsegment with a reagent.

It is also preferred that the first feed has an interruptible fluidic connection line to the second feed. This makes it possible to use the first feed and the second feed to circulate a fluid stored in the diffusion chamber.

The method for investigating cells, in particular mammalian cells in the microfluidic apparatus, comprises introducing cells, in particular mammalian cells, through the first feed, in particular in the form of a cell suspension. The cells, in particular mammalian cells, are lysed in the diffusion chamber.

In principle, it is possible to carry out mixing processes within the diffusion chamber, in particular to mix the mammalian cells with lysis reagents, by vibrating or jerking the diffusion chamber when the feeds are closed. In a first embodiment of the method, however, it is provided that a microfluidic apparatus is used, the first feed of which is connected to the second feed by means of an interruptible fluidic connection line. Lysing is carried out by introducing at least one lysis reagent into the diffusion chamber, in particular through the first feed. In order to mix the mammalian cells and the lysis reagent well and thus achieve rapid lysis, the contents of the diffusion chamber are discharged from the diffusion chamber through the first feed after the lysis reagent has been introduced and returned to the diffusion chamber through the connecting line and the second feed.

In this embodiment of the method, it may be provided in particular that a viability determination of the mammalian cells is to be carried out. For this purpose, after the mammalian cells have been introduced and before lysis by the first feed, reagents for viability determination are introduced into the diffusion chamber and the viability determination is then carried out. These reagents preferably comprise a fluorophore dye that selectively stains dead cells and/or a fluorophore dye that selectively stains living cells. The fluorophore dye may in particular be conjugated with an antibody, linked to a nanoparticle, or bind to free DNA. If two fluorophore dyes are used for living and dead cells, fluorophores that emit at different wavelengths are used for this purpose. The total fluorescence of both fluorophore dyes can be measured to determine viability. If samples from a cell culture are examined in this way several times in succession, changes in the ratio of fluorescence intensity at the two wavelengths can indicate the progress of cultivation.

In addition to determining the viability of the mammalian cells, the culture medium can be analyzed for metabolic products in order to obtain information about the metabolic activity of the mammalian cells. The metabolic products may be glucose, lactate and/or lactate dehydrogenase (LDH) in particular.

In a further embodiment of the method, the method comprises introducing sucrose into the diffusion chamber through the first feed and mixing the sucrose with the lysed mammalian cells to obtain a lower phase comprising sucrose and nucleic acids, in particular DNA and/or RNA. The sucrose can be introduced before or after the introduction of the mammalian cells. In particular, the lysing of the mammalian cells can be carried out using the method according to the first embodiment before the lysate is subsequently mixed with sucrose.

CRISPR-Cas reagents are introduced into the diffusion chamber through the third feed. In this way, an upper phase, in particular one free of sucrose, is obtained, which is arranged above the lower phase in the diffusion chamber. This is followed by a diffusion of nucleic acid molecules from the lower phase to the upper phase and an analysis of the nucleic acid molecules using CRISPR-Cas-based diagnostics. This process exploits the fact that small nucleic acid fragments diffuse more quickly from the sucrose-containing lower phase to the upper phase than larger molecules. For example, this enables quantitative genetic analysis using time-resolved fluorescence detection in a CRISPR-Cas12a system or CRISPR-Cas13 system. The method thus makes it possible to check whether the quality of a cell culture changes over time in terms of viability and, depending on the genetic marker examined, for example in terms of cell composition, expression of tumor markers or mutations, and whether this leads to deviations from the native tumor immune microenvironment. In particular, cellular nucleic acids can be analyzed for genetic tumor markers or single nucleotide polymorphisms (SNPs). This information enables a tumor model to be adapted in a timely manner. In addition to quality control, the method can also be used for endpoint analysis of drugs, for efficacy tests, or for tests on drug combinations in order to analyze phenotypic and genetic characteristics from the same sample material. Another advantage is that data from model generation is comparable with endpoint analyses, as it is based on the same automated procedure.

If there is a high concentration of lysed mammalian cells or a high frequency of a target sequence in the lower phase, it may be sufficient for the upper phase to rest directly on the lower phase. While the upper phase is sucrose-free, the lower phase in this case preferably contains 10 wt % to 40 wt % sucrose.

If, on the other hand, it is necessary to increase the nucleic acid concentration for reliable analysis of the nucleic acid molecules, a sucrose solution containing amplification reagents is preferably introduced into the diffusion chamber through the second feed before introducing the CRISPR-Cas reagents. This results in a middle phase located between the lower phase and the upper phase. The sucrose concentration of the middle phase is lower than the sucrose concentration of the lower phase. In particular, the sucrose concentration in the lower phase is in the range of 30 wt % to 40 wt % and in the middle phase in the range of 10 wt % to 20 wt %, while the upper phase is also sucrose-free in this process. The amplification reagents can in particular be reagents for RPA (recombinase polymerase amplification). They also contain in particular a buffer system. The nucleic acid molecules then diffuse from the lower phase through the middle phase into the upper phase. For an especially isothermal amplification of the nucleic acid molecules in the middle phase, the diffusion chamber is preferably temperature-controlled to a temperature in the range of 20° C. to 42° C.

To implement the method in the microfluidic apparatus, the volume of each phase is preferably in the range of 10 μl to 30 μl, independently of each other. In particular, the volumes are identical.

### BRIEF DESCRIPTION OF THE DRAWINGS

Exemplary embodiments of the invention are shown in the drawings and explained in more detail in the following description.

FIG. 1 schematically shows a microfluidic apparatus according to one exemplary embodiment of the invention.

FIG. 2 shows a transparent isometric illustration of a diffusion chamber of a microfluidic apparatus according to an exemplary embodiment of the invention.

FIG. 3 shows a flowchart of an exemplary embodiment of the method according to the invention.

FIG. 4 shows a schematic illustration of the diffusion chamber of a microfluidic apparatus according to an exemplary embodiment of the invention in a step of an exemplary embodiment of the method according to the invention.

FIG. 5 shows a schematic illustration of a diffusion chamber of a device according to an exemplary embodiment of the invention in another step of an exemplary embodiment of the method according to the invention.

FIG. 6 shows a flowchart of another exemplary embodiment of the method according to the invention.

FIG. 7 shows a schematic illustration of a diffusion chamber of an exemplary embodiment of the microfluidic apparatus in one step of an exemplary embodiment of the method according to the invention.

FIG. 8 shows a schematic illustration of a diffusion chamber of a microfluidic apparatus according to an exemplary embodiment of the invention in another step of an exemplary embodiment of the method according to the invention.

FIG. 9 shows a schematic illustration of a diffusion chamber of a microfluidic apparatus according to an exemplary embodiment of the invention in yet another step of a method according to an exemplary embodiment of the invention.

FIG. 10 shows a schematic illustration of a diffusion chamber of an exemplary embodiment of the microfluidic apparatus in yet another step of a method according to an exemplary embodiment of the invention.

FIG. 11 shows a schematic illustration of a diffusion chamber of an exemplary embodiment of the microfluidic apparatus in a step of another exemplary embodiment of the method according to the invention.

FIG. 12 shows a transparent isometric illustration of a diffusion chamber of a microfluidic apparatus according to another exemplary embodiment of the invention.

FIG. 13 shows a schematic illustration of a diffusion chamber according to another exemplary embodiment of the invention in a step of the method according to the invention.

### EXEMPLARY EMBODIMENTS OF THE INVENTION

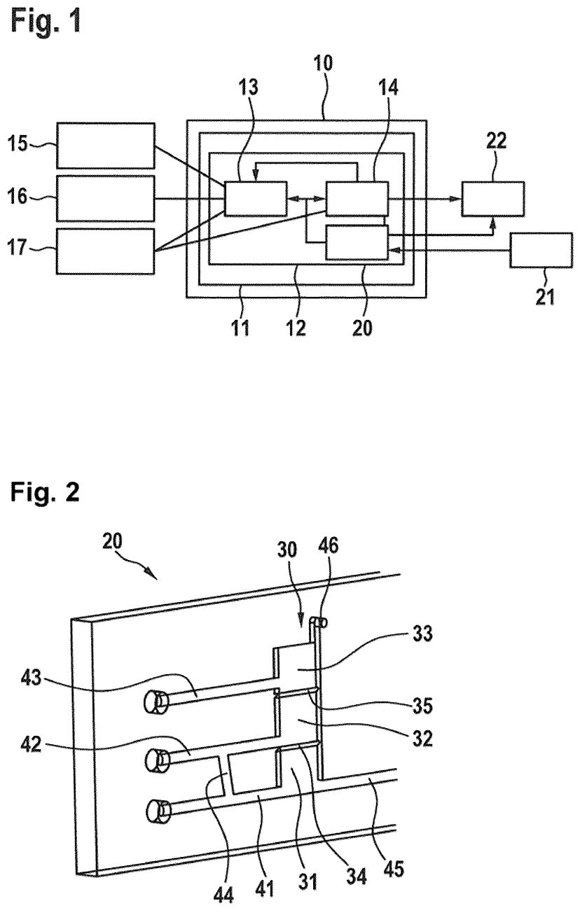

A microfluidic apparatus 10 according to an exemplary embodiment of the invention is shown in FIG. 1. It has a heater 11 and a microfluidic chip 12. A cell culture element 13 is arranged on the microfluidic chip 12, which is designed as a 3D cell culture chamber. This is fluidically connected to a processing element 14, which is set up to carry out cell separation and splitting processes. An atmosphere control element 15 is connected to the cell culture element 13. It enables the microenvironment in cell culture element 13 to be monitored by determining the oxygen concentration, carbon dioxide concentration, and pH value therein. In addition, the atmosphere control element 15 enables the viability of the cell culture to be monitored via its oxygen consumption. A feed 16 supplies the cell culture element 13 with reagents, media, and cells. A microscopic control element 17 monitors the morphology of cells in the cell culture element 13 and in the processing element 14. Cell samples can be fed from the cell culture element 13 or the processing element 14 to an analysis element 20. Furthermore, reagents can be supplied to the analysis element 20 from a reagent reservoir 21. Waste products from the analysis element 20 and the processing element 14 can be collected in a waste reservoir 22.

FIG. 2 shows the structure of the analysis element 20. It has a diffusion chamber 30, which is divided into three segments 31 to 33 arranged one above the other. A first horizontal web 34 is arranged between the lower first segment 31 and the middle second segment 32. A second horizontal web 35 is arranged between the second segment 32 and the upper third segment 33. A first feed 41 opens into the first segment 31. It is fluidically connected to the cell culture element 13 and to the reagent reservoir 21. A second feed 42 opens into the second segment 32 immediately above the first web 34. A third feed 43 opens into the third segment 33 immediately above the second web 35. The second feed 42 and the third feed 43 are fluidically connected to different reagent tanks of the reagent reservoir 21. A connecting line 44 connects the first feed 41 to the second feed 42. A drain 45 at the bottom of the first segment 31 is fluidically connected to the waste reservoir 22. Valves not shown are designed to interrupt the connection of the three feeds 41 to 43 and the outlet 45 to the diffusion chamber 30. Further valves, not shown, are provided to interrupt the connecting line 44 at both ends. A vent opening 46 is arranged on the top surface of the diffusion chamber 30, which has a filter.

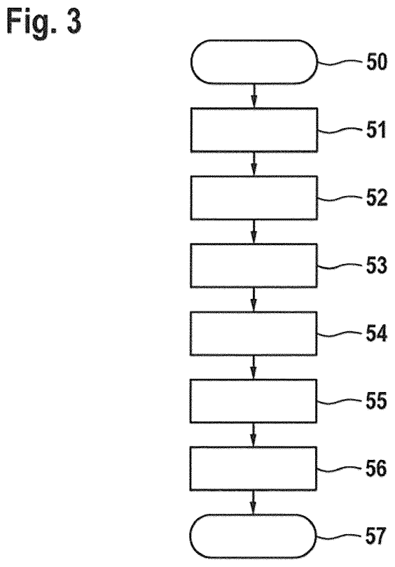

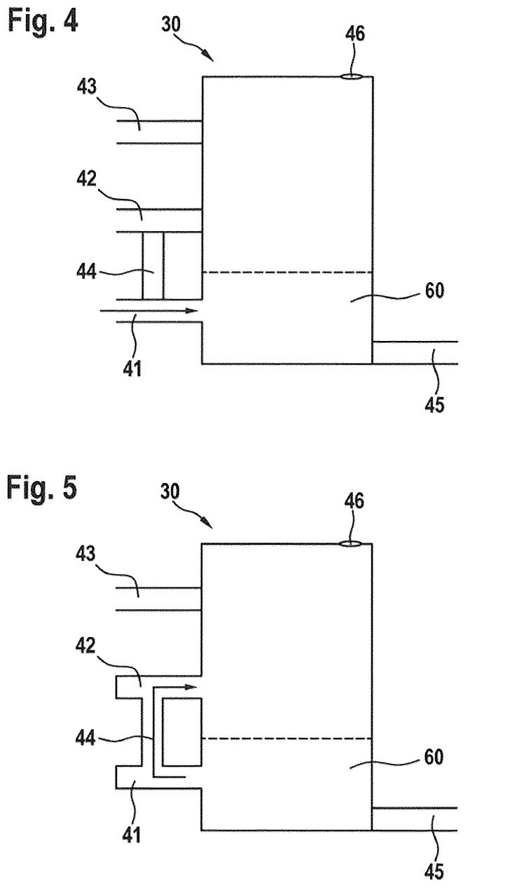

The sequence of a first exemplary embodiment of the method according to the invention is illustrated in FIG. 3. After the start 50 of the process, a suspension of cells from the cell culture element 13 or processing element 14 is first introduced 51 through the first feed 41 into the diffusion chamber 30. As shown in FIG. 4, the first segment 31 of the diffusion chamber 30 is filled with a liquid phase 60. With the exception of the valve which opens the fluidic connection of the feed 41 to the diffusion chamber 30, all other valves are closed. Reagents for viability determination are then introduced 52 into the diffusion chamber 30 through one of the feeds 41-43. Subsequently, viability determination 53 is carried out by means of fluorescence detection. This is possible because the diffusion chamber 30 is formed in a transparent substrate, which in the present exemplary embodiment is polycarbonate. A lysis reagent is now introduced 54 into the diffusion chamber 30 through the second feed 42 or the third feed 43. To mix the liquid phase 60 with the freshly introduced lysis reagent, the connections of the first feed 41 and the second feed 42 to the diffusion chamber are opened, and the connection of the first feed 41 to the cell culture element 13 and the connection of the second feed 42 to the reagent reservoir 21 are closed. Furthermore, the connections of the first feed 41 and the second feed 42 to the connecting line 44 are opened. The liquid phase 60 is now circulated 55 by being pumped out of the first segment 31 of the diffusion chamber 30 through the first feed line 41, passed through the connecting line 44 into the second feed line 42 and fed back into the second segment 32 of the diffusion chamber 30, from where it flows back into the first segment 31 under the action of gravity. This is illustrated in FIG. 5. This circulation thoroughly mixes all components of the liquid phase 60 with each other so that the cells contained in the liquid phase 60 can then be lysed 56 to release the nucleic acids contained therein. If necessary, a sample of the nucleic acid solution can be taken via the drain 45 for further analysis. The process is then terminated 57.

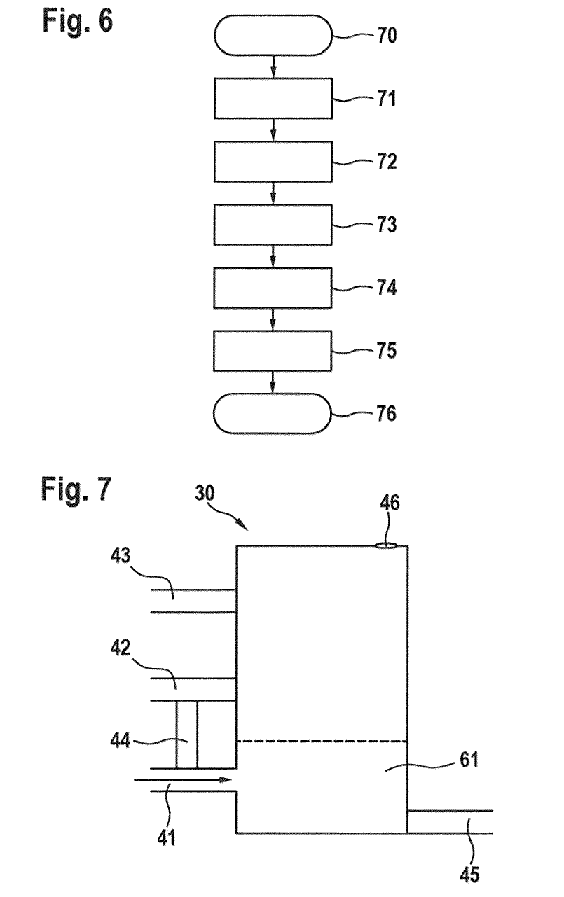

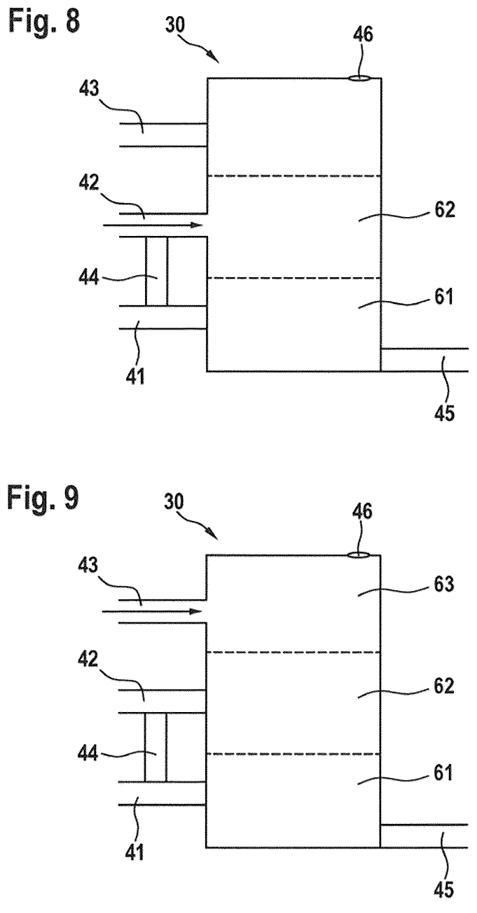

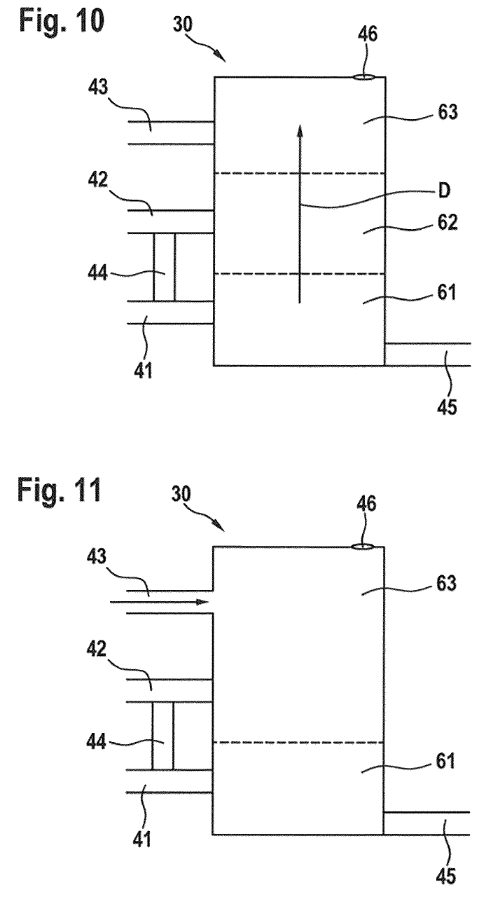

The sequence of a second exemplary embodiment of the method according to the invention is shown in FIG. 6. After the start 70 of the method, cells are introduced 71 from the cell culture element 13 into the first segment 31 of the diffusion chamber 30. These cells are then lysed. Lysis can be carried out by means of steps 54 to 56 of the first exemplary embodiment of the method according to the invention, or a different lysis process can also be provided. This is followed by the introduction 72 of sucrose through the first feed 41 into the diffusion chamber 30, so that a lower phase 61 is obtained which contains, for example, 40% by weight of sucrose. This is illustrated in FIG. 7. The connection of the first feed 41 to the diffusion chamber 30 is now closed, and the connection of the second feed 42 to the diffusion chamber 30 is opened. Through this, sucrose and amplification reagents are introduced from the reagent reservoir 21 into the second segment 32 of the diffusion chamber 30 in order to obtain a middle phase 62 containing 10% by weight of sucrose 73. This is illustrated in FIG. 8. After closing the connection of the second feed 42 to the diffusion chamber 30 and opening the connection of the third feed 43 to the diffusion chamber 30, CRISPR-Cas reagents are introduced 74 from the reagent reservoir 21 into the third segment 33 of the diffusion chamber 30 through the third feed 43. This produces an upper phase 63. This is illustrated in FIG. 9. After closing the connection of the third feed 43 to the diffusion chamber 30, an analysis 75 of nucleic acid molecules from the lysed cells is performed using CRISPR-Cas-based diagnostics. This involves diffusion D of the nucleic acid molecules from the lower phase 61 into the middle phase 62, where they are amplified isothermally, and then further into the upper phase 63, where they can be subjected to fluorescence analysis. The nucleic acid molecules follow the concentration gradient of sucrose in the three phases 61 to 63.

In a third exemplary embodiment of the method according to the invention, the second exemplary embodiment is modified by omitting step 73. As shown in FIG. 11, the lower phase 61 is immediately superimposed on the upper phase 63 without first generating a middle phase 62. Nucleic acid molecules diffuse from the lower phase 61 into the upper phase 63 without being amplified. However, if the concentration of nucleic acid molecules in the lower phase is sufficiently high, a sufficiently accurate fluorescence analysis of the nucleic acid molecules in the CRISPR-Cas system can also be performed in the third exemplary embodiment of the method.

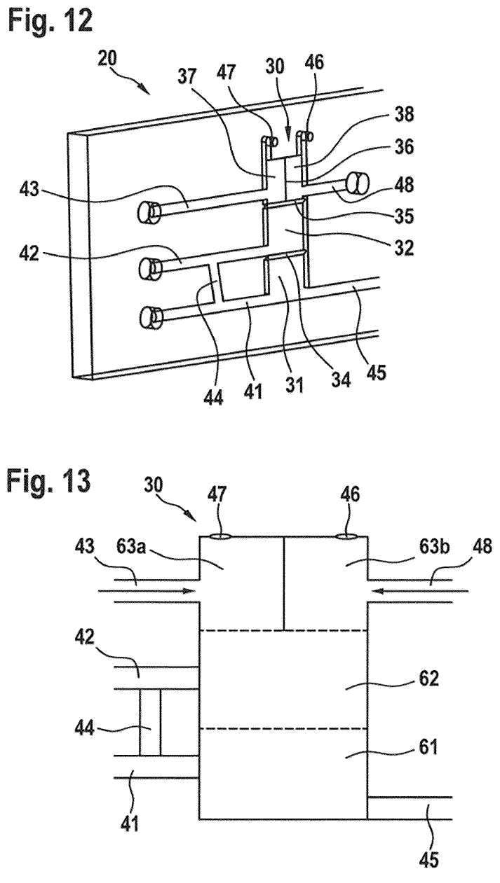

FIG. 12 shows the structure of the analysis element 20 in a second exemplary embodiment of the microfluidic apparatus 10. The diffusion chamber 30 differs from the first exemplary embodiment in that the third segment 33 is divided into two subsegments 37, 38 by a horizontal partition wall 36. The diffusion chamber 30 has two vent openings 46, 47 on its top surface, wherein each of the subsegments 37, 38 has one of the vent openings 46, 47. While the third feed 43 opens into the first subsegment 37, a further third feed 48 opens into the second subsegment 38. The second exemplary embodiment of the microfluidic apparatus allows a modified implementation of the second exemplary embodiment of the method. As shown in FIG. 13, the two third feeds 43, 48 generate two different upper phases 63a, 63b by introducing different CRISPR-Cas systems into the subsegments 37, 38. This makes it possible to examine the lysed cells simultaneously for different targets.

In all exemplary embodiments of the microfluidic apparatus 10 and in all exemplary embodiments of the method according to the invention, after completion of an analysis, the entire contents of the diffusion chamber can be discharged through the outlet 45 into the waste reservoir 22. The feeds 41 to 43 and, if applicable, 48, as well as the interior of the diffusion chamber 30, can be flushed with a flushing liquid from the reagent reservoir 21 in order to prepare the analysis element 20 for its next use. In this way, samples can be taken from the cell culture element 13 at regular intervals and examined in the analysis element 20 in a timely manner.

1. A microfluidic apparatus, comprising:
multiple feeds including at least one first feed, at least one second feed, and at least one third feed; and
a diffusion chamber into which the multiple feeds open,
wherein the at least one first feed opens into the diffusion chamber below at the least one second feed, and
wherein the at least one third feed opens into the diffusion chamber above the second feed.

2. The microfluidic apparatus according to claim 1, further comprising a cell culture element and a reagent reservoir, wherein:
the at least one first feed is fluidically connected to the cell culture element and,
the at least one second feed and the at least one third feed are each fluidically connected to the reagent reservoir.

3. The microfluidic apparatus according to claim 1, wherein the diffusion chamber has at least one vent opening on its top surface.

4. The microfluidic apparatus according to claim 1, characterized in that wherein:
the at least one first feed opens into a first segment of the diffusion chamber,
the at least one second feed opens into a second segment of the diffusion chamber, and
the at least one third feed opens into a third segment of the diffusion chamber.

5. The microfluidic apparatus according to claim 4, further comprising a first web is arranged between the at least one first segment and the at least one second segment, and a second web arranged between the at least one second segment and the at least one third segment.

6. The microfluidic apparatus according to claim 4, wherein the at least one third segment is divided into several subsegments by means way of at least one partition, wherein a respective third feed of the at least one third feed opens into each subsegment.

7. The microfluidic apparatus according to claim 1, wherein the at least one first feed has an interruptible fluidic connection line to the at least one second feed.

8. A method for investigating mammalian cells in the microfluidic apparatus according to claim 1, comprising introducing mammalian cells into the diffusion chamber through the at least one first feed, and lysing the mammalian cells.

9. The method according to claim 8 using a microfluidic apparatus, wherein the lysing is carried out by:
introducing at least one lysis reagent into the diffusion chamber; and
discharging the contents of the diffusion chamber from the diffusion chamber through the at least one first feed and returning the contents to the diffusion chamber through the connecting line and the at least one second feed.

10. The method according to claim 8, further comprising:
after introducing the mammalian cells and before lysing by the at least one first feed inlet, introducing reagents for viability determination into the diffusion chamber; and
carrying out viability determination.

11. The method according to claim 8, further comprising:
introducing sucrose into the diffusion chamber through the at least one first feed,
mixing the sucrose with the lysed mammalian cells to obtain a lower phase containing sucrose and nucleic acids,
introducing CRISPR-Cas reagents into the diffusion chamber through the at least one third feed to obtain an upper phase,
diffusing nucleic acid molecules from the lower phase to the upper phase, and
analyzing nucleic acid molecules by CRISPR-Cas based diagnostics.

12. The method according to claim 11, further comprising:
before the CRISPR-Cas reagents are introduced, introducing a sucrose solution containing amplification reagents into the diffusion chamber through the at least one second feed in order to obtain a middle phase, wherein a sucrose concentration of the middle phase is lower than a sucrose concentration in the lower phase.

13. The method according to claim 11, wherein a volume of each phase is in the range of 10 μl to 30 μl.

14. The microfluidic apparatus according to claim 1, further comprising (i) a processing element configured to separate and split mammalian cells, and (ii) a reagent reservoir, wherein:
the at least one first feed is fluidically connected to the processing element, and
the at least one second feed and the at least one third feed are each fluidically connected to the reagent reservoir.
